(19) United States
(12) Patent Application Publication
(10) Pub. No.: US 2026/0354865 A1
(43) Pub. Date: Nov. 12, 2026

(54) BONE STAPLE AND TECHNIQUES FOR FIXATING AN AKIN OSTEOTOMY

(71) Applicant: Treace Medical Concepts, Inc., Ponte Vedra, FL (US)

(72) Inventors: Jason May, St. Johns, FL (US); Jared White, St. Augustine, FL (US); Amber M. Shane, Orlando, FL (US); Robert D. Santrock, Morgantown, WV (US); Mark E. Easley, Durham, NC (US)

(21) Appl. No.: 19/578,749

(22) Filed: Mar. 25, 2026

Related U.S. Application Data
(60) Provisional application No. 63/778,062, filed on Mar. 26, 2025.

(51) Int. Cl. A61B 17/56; A61B 17/00; A61B 17/064

(52) U.S. Cl. CPC A61B 17/56; A61B 17/0642; A61B 2017/00477; A61B 2017/565

(57) ABSTRACT

A system can include a staple and an inserter. The staple can include a bridge connecting a first leg and a second leg, first and second handling couplings extending through a top surface of the staple, and a bottom surface configured to face one or more bones. The bridge can include a convex bottom surface that extends along at least a portion of the bottom surface of the staple at the bridge. The inserter can be configured to couple to the first and second handling couplings at the staple. When the inserter is connected to the staple, the inserter can be configured to apply a load force at the staple to cause the first leg and the second leg to move apart from one another while maintaining the convex bottom surface extending along at least a portion of the bridge.

### CROSS REFERENCE TO RELATED APPLICATIONS

This application claims priority to and the benefit of U.S. Provisional Patent Application No. 63/778,062, filed Mar. 26, 2025, the entire contents of which is hereby incorporated by reference.

### TECHNICAL FIELD

This disclosure relates to Akin osteotomies and, more particularly, to implants and techniques for fixating bones after performing an Akin osteotomy

### BACKGROUND

The human foot includes the five toes (which are also known as the “phalanges”) and their connecting long bones (or “metatarsals”). The joint between a metatarsal and a phalange is called the metatarsophalangeal (“MTP”) joint. Several small bones together comprise a phalanx or toe. Four of the five toes have three phalanx bones respectively connected by two joints. The big toe (or “hallux”) has two phalanx bones distal and proximal with a joint in between called the interphalangeal joint. The phalanges are connected to the metatarsals at the ball of the foot.

An Akin osteotomy is a surgical procedure, often used in conjunction with other bunion correction procedures, that involves making a medial closing wedge osteotomy on the proximal phalanx of the big toe. After cutting and removing a wedge-shaped section of bone, the wedge-shaped opening is closed. An implant can be applied to hold the wedge closed.

### SUMMARY

In general, this disclosure is directed to staple implant devices for closing an Akin osteotomy and associate techniques. In some implementations, a clinician surgically accesses the first proximal phalanx of the foot and performs an Akin osteotomy on the proximal phalanx. The Akin osteotomy can involve cutting a wedge-shaped section of bone out of the proximal phalanx, with the base or large portion of the wedge on the medial side of the bone and the tip or narrow portion of the wedge on a lateral portion of the foot. The wedge may or may not extend through the lateral-most side of the proximal phalanx and is typically performed to leave a small bridge of bone on the lateral-most side of the proximal phalanx at the tip of the wedge. The small bridge of bone can function as a hinge about which the cut wedge is subsequently closed. The clinician can close the wedge-shaped opening formed by cutting and removing the wedge-shaped bone portion. Closing the wedge can cause the distal portion of the proximal phalanx (distal of the wedge opening) and distal phalanx attached to the proximal phalanx to shift medially in the transverse plane. After closing the wedge, the clinician can apply an implant as described herein to the proximal phalanx.

As described in greater detail below, techniques according to the disclosure can utilize a staple specifically configured to be applied to fixate an Akin osteotomy. The staple can include a first leg insertable into the proximal phalanx at a location proximal of the Akin osteotomy, a second leg insertable into the proximal phalanx at a location distal of the Akin osteotomy, and bridge spanning the closed Akin osteotomy. The bridge can be specifically configured to conform to the medial and/or dorsal surface of the proximal phalanx across the closed Akin osteotomy. This can provide an implant with reduced medial prominence that can promote healing and reduce patient discomfort following surgery.

In some examples, a bone staple can include one or more features that configure the body of the staple to anatomically fit a target implant region. In the noted exemplary application following an Akin osteotomy, such a bone staple can include a geometric profile at the bridge corresponding to the target implant region anatomical surface contouring. This conforming geometric profile at the bridge can define one or more surfaces that, in two or more planes, change in elevation along the body of the staple in a manner that can mimic the target implant region anatomical surface contouring. In this way, the staple can provide a more robust bone fixation when implanted at that corresponding target implant region anatomical surface contouring by better conforming to the target bone surfaces where the staple is to be implanted. In the noted exemplary application following an Akin osteotomy, the staple body can include one or more regions, such as at the bridge, conforming to the anatomical bone surface contour at a metaphysis portion of the proximal phalanx. In this application, this staple can bridge across the cut interface between the re-aligned proximal portion of the proximal phalanx and distal portion of the proximal phalanx in a way that conforms to the anatomical bone surface contour at a metaphysis portion of the proximal portion and a metaphysis portion of the distal portion of the proximal phalanx. This conformance to the particular anatomical bone surface contour can enable more efficient transfer of fixation and/or stabilization forces from the staple to the bone portions fixated relative to one another via the staple.

One embodiment disclosed herein includes a system. This system embodiment can include a staple and an inserter. The staple can include a first leg on a first side of the staple, a second leg on a second side of the staple, a bridge connecting the first leg and the second leg, a first handling coupling extending through a top surface of the staple on the first side of the staple, and a second handling coupling extending through the top surface of the staple on the second side of the staple. The staple can further include a bottom surface configured to face one or more bones, with the top surface of the staple being opposite the bottom surface of the staple. The bridge can include a convex bottom surface that extends along at least a portion of the bottom surface of the staple at the bridge. The inserter is configured to couple to the first handling coupling through the top surface of the staple and to the second handling coupling through the top surface of the staple. When the inserter is connected to the first handling coupling and to the second handling coupling, the inserter is configured to apply a load force at the staple to cause the first leg and the second leg to move apart from one another.

In a further embodiment of this staple, the convex bottom surface at the bridge can correspond to a curvature at a surface of a phalanx bone. For example, the convex bottom surface at the bridge can match a concave curvature at a surface of a metaphysis portion of the phalanx bone. In some such examples, the convex bottom surface can define a local maximum point at a central portion along a length of the bridge, and this local maximum point can be along the length of the bridge so as to be spaced equally from each of the first handling coupling and the second handling. In additional or alternative examples, a length of the bridge between the first leg and the second leg can be configured to span across a cut at the surface of the metaphysis portion of the phalanx bone. For instance, the inserter can be configured such that when the inserter is removed from at least one of the first handling coupling and the second handling coupling the load force is removed from the staple to cause the first leg and the second leg to move toward one another to apply a compression force. The staple can be configured such that the convex bottom surface at the bridge is present both when the load force is applied and when the load force is removed. The staple can further include a concave top surface extending along at least a portion of the top surface of the staple at the bridge, and the staple can be configured such that each of the convex bottom surface at the bridge at the concave top surface at the bridge is present both when the load force is applied and when the load force is removed.

In a further embodiment of this staple, the bottom surface of the staple at the bridge is configured to directly contact a surface of a metaphysis portion of a phalanx bone without any inserter structure between the bottom surface of the staple and the surface of the metaphysis portion of the phalanx bone. For example, the first handling coupling can extend into the first leg, the second handling coupling can extend into the second leg, the inserter can be configured to couple to the first handling coupling within the first leg and to the second handling coupling within the second leg. In some such examples, the first handling coupling can be configured to receive the inserter without the inserter extending under the bottom surface of the staple, and the second handling coupling can be configured to receive the inserter without extending under the bottom surface of the staple. The inserter could include comprises a first coupling shaft configured to connect to the first handling coupling through the top surface of the staple and a second coupling shaft configured to connect to the second handling coupling through the top surface of the staple. And, the inserter can be configured to apply the load force when the first coupling shaft and the second coupling shaft are biased toward each other. When the first coupling shaft is connected to the first handling coupling and the second coupling shaft is connected to the second handling coupling, the first coupling shaft and the second coupling shaft can be configured to move toward each other to apply the load force at the staple, and the first coupling shaft and the second coupling shaft can be configured to move away from each other to move the first leg of the staple and the second leg of the staple toward one another.

Another system embodiment disclosed herein includes a staple, first and second coupling shafts, and a connector. The staple can include a first leg on a first side of the staple, a second leg on a second side of the staple, a bridge connecting the first leg and the second leg, a first handling coupling extending through a top surface of the staple on the first side of the staple, and a second handling coupling extending through the top surface of the staple on the second side of the staple. The staple has a bottom surface configured to face one or more bones, and the top surface of the staple is opposite the bottom surface of the staple. The bridge includes a convex bottom surface that extends along at least a portion of the bottom surface of the staple at the bridge. The first coupling shaft is configured to couple to the first handling coupling, the second coupling shaft configured to couple to the second handling coupling, and the connector is configured to join the first coupling shaft and the second coupling shaft. When the connector is removed from at least one of the first coupling shaft and the second coupling shaft, the staple is configured such that the first leg and the second leg move toward one another while maintaining the convex bottom surface at the bridge.

In a further embodiment of this system, when the connector is removed from at least one of the first coupling shaft and the second coupling shaft, the staple is configured such that the first leg and the second leg extend parallel to one another while maintaining the convex bottom surface at the bridge.

Another embodiment disclosed herein is a staple. This staple embodiment can include a top surface, a bottom surface opposite the top surface, with the bottom surface configured to face one or more bones when the staple is implanted at the one or more bones, a first leg at a first side of the staple, a second leg at a second, opposite side of the staple, a bridge connecting the first and second legs, the bridge comprising a convex bottom surface that extends along at least a portion of the bottom surface of the staple at the bridge, a first handling coupling extending through the top surface of the staple at the first side of the staple, the first handling coupling configured to receive a first coupling shaft at the top surface of the first side of the staple; and a second handling coupling extending through the top surface of the staple at the second side of the staple, the second handling coupling configured to receive a second coupling shaft at the top surface of the second side of the staple.

In a further embodiment of this staple, the first handling coupling and the second handling coupling can each threaded, the first handling coupling can be configured to receive the first coupling shaft though the top surface of the first side of the staple to engage threading at the first handling coupling, and the second handling coupling is configured to receive the second coupling shaft though the top surface of the second side of the staple to engage threading at the second handling coupling. For example, the first leg can include a first set of teeth extending partially around a perimeter of the first leg and facing the second leg, and the second leg can include a second set of teeth extending partially around a perimeter of the second leg and facing the first leg.

Another embodiment disclosed herein includes a method of fixating one or more bones. This method embodiment can include the step of: positioning a first leg of a staple connected to an inserter in a first implant hole of a first bone portion and a second leg of the staple connected to the inserter in a second implant hole of a second bone portion, wherein the first leg of the staple is connected to the second leg of the staple by a bridge, wherein the staple is positioned such that a bottom surface of the staple faces the first bone portion and the second bone portion and a top surface of the staple faces away from the first bone portion and the second bone portion, wherein the bridge includes a convex bottom surface that extends along at least a portion of the bottom surface of the staple at the bridge, wherein the staple is positioned with the convex bottom surface at each of the first bone portion and the second bone portion, and wherein the inserter is connected to the staple through the top surface of the staple. And, this method embodiment can include the step of: detaching the inserter from the staple.

In a further embodiment of this method, the method includes an additional step of: prior to positioning the staple and prior to detaching the inserter, performing an Akin osteotomy at the first bone portion and the second bone portion.

In a further embodiment of this method, the staple is positioned with the convex bottom surface at each of a concave curvature at a surface of a metaphysis portion of the first bone portion and a concave curvature at a surface of a metaphysis portion of the second bone portion. For example, the first bone portion can be a first portion of a phalanx bone and the second bone portion can be a second portion of the phalanx bone, with the first portion of the phalanx bone separated from the second portion of the phalanx bone by a cut interface. For such example, the staple can be positioned such that the convex bottom surface at the bridge extends from the first portion of the phalanx bone to the second portion of the phalanx bone and across the cut interface. For instance, the convex bottom surface at the bridge can define a local maximum point, and the staple can be positioned such that the local maximum point of the convex bottom surface at the bridge is at least the cut interface.

In a further embodiment of this method, the method can include a step of: applying a load force at the staple using the inserter, wherein, as the load force is applied at the staple using the inserter, the first leg and the second leg are caused to move apart from one another. The method can further include a step of: removing the load force from the staple (i) after positioning the first leg in the first implant hole and the second leg in the second implant hole, (ii) after positioning the convex bottom surface at the bridge at each of the first bone portion and the second bone portion, and (iii) prior to detaching the inserter from the staple. For example, removing the load force from the staple can cause the first leg and the second leg to move toward one another to apply a compression force at the first bone and the second bone while maintaining the convex bottom surface at each of the first bone portion and the second bone portion. The inserter can include a first coupling shaft connected through the top surface of the staple at the first leg and a second coupling shaft connected through the top surface of the staple at the second leg, and applying the load force comprises biasing the first coupling shaft and the second coupling shaft toward each other. For instance, a connector can join the first coupling shaft and the second coupling shaft to bias the first coupling shaft and the second coupling shaft toward each other and maintain the load force at the staple while the connector joins the first coupling shaft and the second coupling shaft. The method could then include an additional step of: removing the connector from the first coupling shaft and the second coupling shaft to cause the first coupling shaft to move away from the second coupling shaft while maintaining the convex bottom surface at the bridge of the staple.

The details of one or more examples are set forth in the accompanying drawings and the description below. Other features, objects, and advantages will be apparent from the description and drawings, and from the claims.

### BRIEF DESCRIPTION OF DRAWINGS

FIGS. 1A and 1B are front views of a foot showing a normal first metatarsal position and an example frontal plane rotational misalignment position, respectively.

FIGS. 2A and 2B are top views of a foot showing a normal first metatarsal position and an example transverse plane misalignment position, respectively.

FIGS. 3A and 3B are side views of a foot showing a normal first metatarsal position and an example sagittal plane misalignment position, respectively.

FIG. 4 is a top view of a portion of a foot showing an example Akin osteotomy.

FIGS. 5A-5C show an embodiment of staple. FIG. 5A is a perspective view of this staple embodiment, FIG. 5B is a top surface plan view of this staple embodiment, and FIG. 5C is an elevational view of this embodiment of the staple.

FIGS. 6A-6C show an embodiment of an inserter coupled to the staple of FIGS. 5A-5C. FIG. 6A is a cross-sectional view of the inserter coupled to the staple, FIG. 6B is a close-up view of the cross-section at FIG. 6A showing the inserter coupled to the staple via handling couplings at the staples, and FIG. 6C is an elevational view of the inserter coupled to the staple to apply a load force to the staple and position the staple across bone portions.

FIG. 7 is a plan view showing the staple of FIGS. 5A-5C positioned at a medial side of, and across a cut interface at, a phalanx bone.

FIG. 8 is an elevational view showing the staple of FIGS. 5A-5C positioned at a dorsal side of, and across a cut interface at, a phalanx bone.

FIG. 9 is a flow diagram of an embodiment of a method for fixating one or more bones, for instance, using the staple of FIGS. 5A-5C.

### DETAILED DESCRIPTION

In general, the present disclosure is directed to staple implant for use in an Akin osteotomy procedure and associated techniques utilizing the staple. In general, an Akin osteotomy is a surgical procedure performed to correct deformities in the foot, particularly hallux valgus (bunion). The procedure may involve a bone cut (osteotomy) at the base of the first proximal phalanx, which can create a cut interface at that first proximal phalanx between a first portion of the first proximal phalanx and a second portion of the first proximal phalanx. The cut is typically done at an angle to remove a wedge-shaped section of bone. The angle of the wedge can be selected based on the amount of bone realignment desired to reduce the angle of the deformity. Realignment following the osteotomy can straighten the great toe, bringing it closer to its anatomical position.

An Akin osteotomy and fusion of the resulting cut bone may be performed for a variety of clinical reasons and indications. An Akin osteotomy and fusion of the proximal phalanx may be performed to treat hallux valgus. An Akin osteotomy and fusion of the proximal phalanx can be performed alone or in combination with other osteotomy and bone realignment procedures (e.g., a first metatarsal osteotomy, a tarsometatarsal joint fusion procedure).

Hallux valgus, also referred to as hallux abducto valgus, is a complex progressive condition that is characterized by lateral deviation (valgus, abduction) of the hallux and medial deviation of the first metatarsophalangeal joint. Hallux valgus typically results in a progressive increase in the hallux abductus angle, the angle between the long axes of the first metatarsal and proximal phalanx in the transverse plane. An increase in the hallux abductus angle may tend to laterally displace the plantar aponeurosis and tendons of the intrinsic and extrinsic muscles that cross over the first metatarsophalangeal joint from the metatarsal to the hallux.

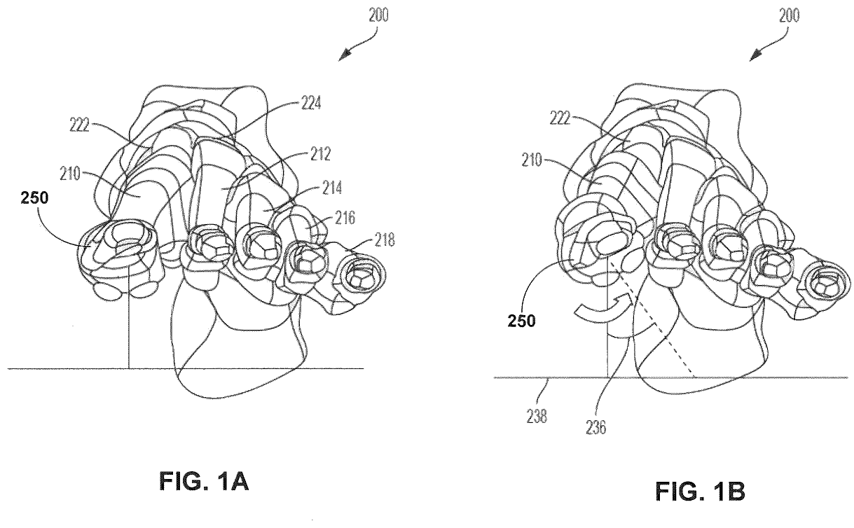

To further understand example techniques and staple implants of the disclosure, the anatomy of the foot will first be described with respect to FIG. 1-3 along with example misalignments that may occur and be corrected according to the present disclosure. A bone misalignment may be caused by hallux valgus (bunion), hallux rigidus, a natural growth deformity, and/or other condition. The condition may present with a misalignment of one or more bones in the foot.

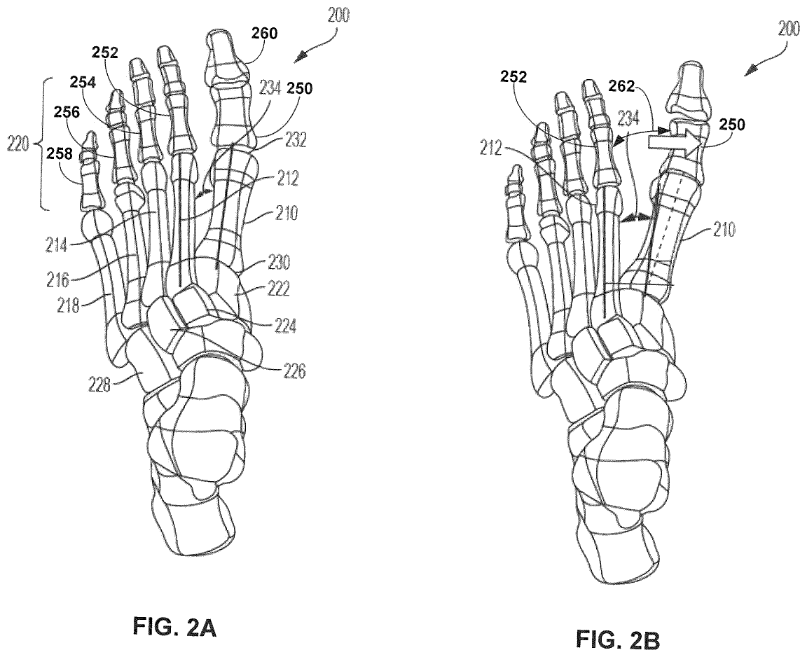

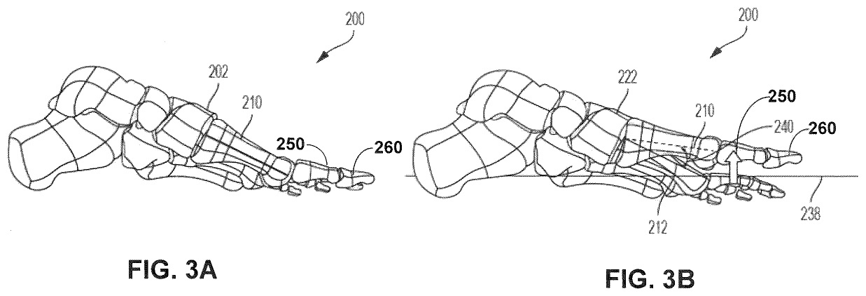

FIGS. 1A and 1B are front views of foot 200 showing a normal first metatarsal position and an example frontal plane rotational misalignment position, respectively. FIGS. 2A and 2B are top views of foot 200 showing a normal first metatarsal position and an example transverse plane misalignment position, respectively. FIGS. 3A and 3B are side views of foot 200 showing a normal first metatarsal position and an example sagittal plane misalignment position, respectively. While FIGS. 1B, 2B, and 3B show each respective planar misalignment in isolation, in practice, a metatarsal may be misaligned in any two of the three planes or even all three planes. Accordingly, it should be appreciated that the depiction of a single plane of misalignment in each of FIGS. 1B, 2B, and 3B is for purposes of illustration and a metatarsal and/or phalanx may be misaligned in multiple planes that is desirably corrected. Further, a bone condition treated according to the disclosure may not present any of the example misalignments described with respect to FIGS. 1B, 2B, and 3B, and it should be appreciated that the disclosure is not limited in this respect.

With reference to FIGS. 1A and 2A, foot 200 is composed of multiple bones including a first metatarsal 210, a second metatarsal 212, a third metatarsal 214, a fourth metatarsal 216, and a fifth metatarsal 218. The metatarsals are connected distally to phalanges 220 and, more particularly, each to a respective proximal phalanx. In particular, the first metatarsal 210 is connected distally to first proximal phalanx 250, the second metatarsal 212 is connected distally to second proximal phalanx 252, the third metatarsal 214 is connected distally to third proximal phalanx 254, the fourth metatarsal 216 is connected distally to fourth proximal phalanx 256, and the fifth metatarsal 218 is connected distally to fifth proximal phalanx 258. The joint 232 between a metatarsal and a corresponding opposed proximal phalanx is referred to as a metatarsophalangeal (“MTP”) joint. The first MTP joint is labeled as joint 232 in FIG. 2A, although second, third, fourth, and fifth MTP joints are also illustrated in series adjacent to the first MTP joint. The first proximal phalanx 250 is connected distally to the first distal phalanx 260 across the proximal interphalangeal joint 262.

The first metatarsal 210 is connected proximally to a medial cuneiform 222, while the second metatarsal 212 is connected proximally to an intermediate cuneiform 224 and the third metatarsal is connected proximally to lateral cuneiform 226. The fourth and fifth metatarsals 216, 218 are connected proximally to the cuboid bone 228. The joint 230 between a metatarsal and respective cuneiform (e.g., first metatarsal 210 and medial cuneiform 222) is referred to as the tarsometatarsal (“TMT”) joint. The angle 234 between adjacent metatarsals (e.g., first metatarsal 210 and second metatarsal 212) is referred to as the intermetatarsal angle (“IMA”).

As noted, FIG. 1A is a frontal plane view of foot 200 showing a typical position for first metatarsal 210. The frontal plane, which is also known as the coronal plane, is generally considered any vertical plane that divides the body into anterior and posterior sections. On foot 200, the frontal plane is a plane that extends vertically and is perpendicular to an axis extending proximally to distally along the length of the foot. FIG. 1A shows first metatarsal 210 in a typical rotational position in the frontal plane. FIG. 1B shows first metatarsal 210 with a frontal plane rotational deformity characterized by a rotational angle 236 relative to ground, as indicated by line 238.

FIG. 2A is a top view of foot 200 showing a typical position of first metatarsal 210 in the transverse plane. The transverse plane, which is also known as the horizontal plane, axial plane, or transaxial plane, is considered any plane that divides the body into superior and inferior parts. On foot 200, the transverse plane is a plane that extends horizontally and is perpendicular to an axis extending dorsally to plantarly (top to bottom) across the foot. FIG. 2A shows first metatarsal 210 with a typical IMA 234 in the transverse plane. FIG. 2B shows first metatarsal 210 with a transverse plane rotational deformity characterized by a greater IMA caused by the distal end of first metatarsal 210 being pivoted medially relative to the second metatarsal 212.

FIG. 3A is a side view of foot 200 showing a typical position of first metatarsal 210 in the sagittal plane. The sagittal plane is a plane parallel to the sagittal suture which divides the body into right and left halves. On foot 200, the sagittal plane is a plane that extends vertically and intersects an axis extending proximally to distally along the length of the foot. FIG. 3A shows first metatarsal 210 with a typical rotational position in the sagittal plane. FIG. 3B shows first metatarsal 210 with a sagittal plane rotational deformity characterized by a rotational angle 240 relative to ground, as indicated by line 238.

Standard medical planes of reference and descriptive terminology are employed in this disclosure. A sagittal plane divides a body into right and left portions. A coronal or frontal plane divides a body into anterior and posterior portions. A transverse plane divides a body into superior and inferior portions. Anterior means toward the front of a body. Posterior means toward the back of a body. Superior or cephalad means toward the head. Inferior or caudal means toward the feet or tail. Medial means toward the midline of a body (e.g., toward a plane of bilateral symmetry of the body). Lateral means away from the midline of a body or away from a plane of bilateral symmetry of the body. Proximal means toward the trunk of the body. Distal means away from the trunk. Dorsal means toward the top of the foot or other body structure. Plantar means toward the sole of the foot or toward the bottom of the body structure.

As briefly discussed above, an Akin osteotomy may be performed to realign the big toe of the foot and help restore the anatomic positioning of the big toe (e.g., to help correct a bunion deformity). During the procedure, the patient may be placed under local or general anesthesia, and the surgical site prepared with a sterile field. A small incision may be made through the skin along the medial side of the first metatarsophalangeal joint 232, providing access to the proximal base of proximal phalanx 250. The clinician can then cut and remove a portion of proximal phalanx 250 to facilitate repositioning of the proximal phalanx and distal phalanx 260.

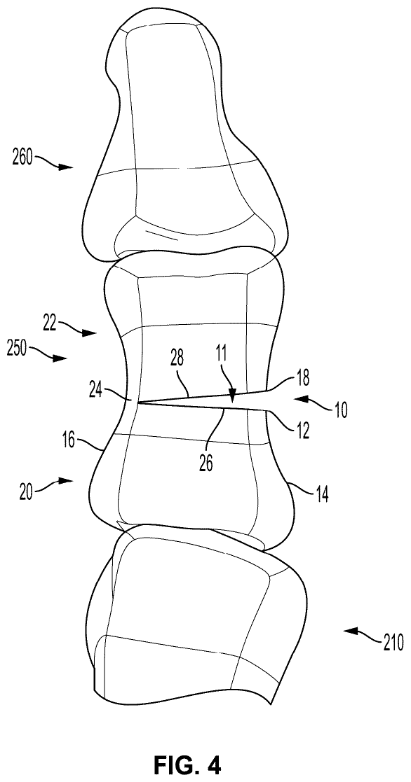

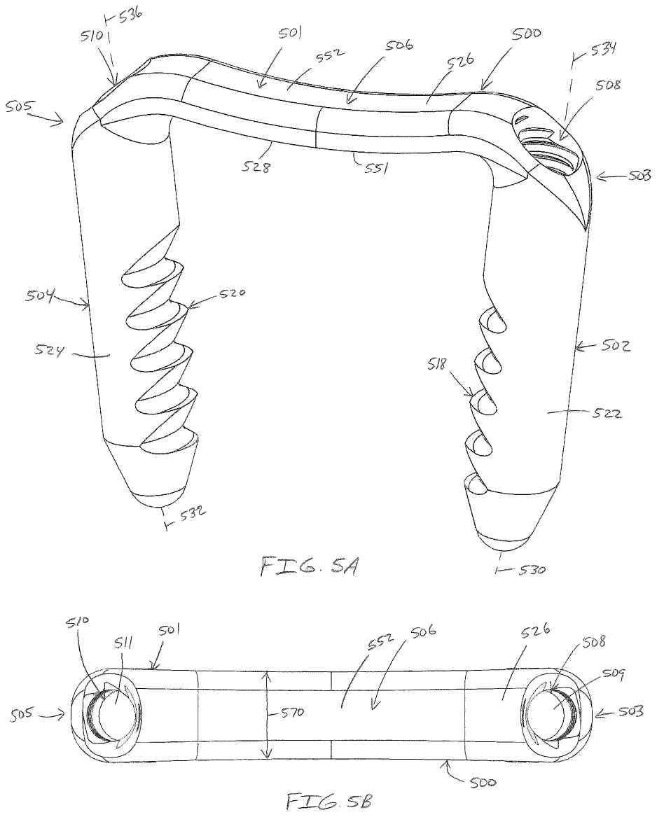

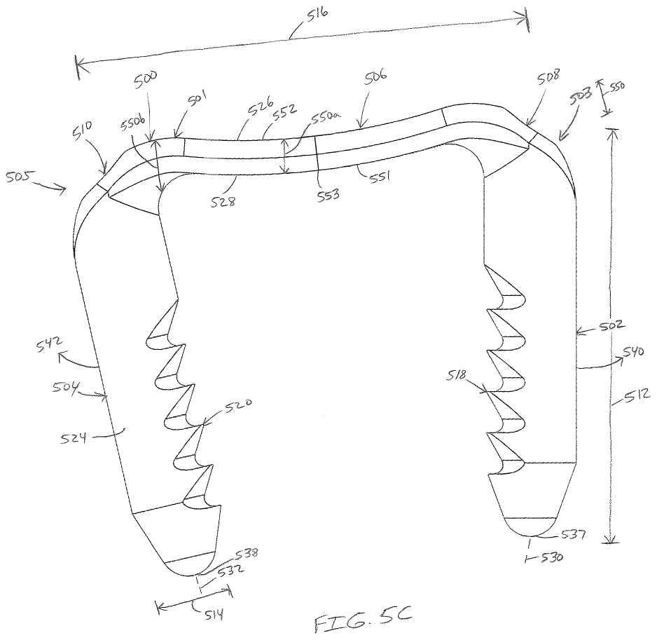

FIG. 4 is a top view of a portion of a foot showing an example Akin osteotomy. As shown in this example, a wedge-shaped opening 10 is cut into proximal phalanx 250. A cutting instrument can be used to form a first cut 12 extending from a medial side 14 of proximal phalanx 250 toward a lateral side 16 of the proximal phalanx. First cut 12 may extend perpendicularly across proximal phalanx 250 or maybe angled in a proximal-to-distal direction along the length of proximal phalanx 250. A second cut 18 can also be formed extending from medial side 14 of proximal phalanx 250 toward lateral side 16 of the proximal phalanx. Second cut 18 can be offset from first cut 12 (e.g., distally along the length of proximal phalanx 250) on the medial side 14 of proximal phalanx 250. The extent of the offset can define the width of the wedge of bone subsequently removed and the extent of correction. Second cut 18 can intersect first cut 12 to form an apex of the wedge-shaped bone portion to be removed as part of the osteotomy. Any suitable cutting instrument can be used to form wedge-shaped opening 10. Example cutting instruments that can be used include, but are not limited to, a saw blade, a rotary bur, and the like.

It should be appreciated that reference to first and second herein is intended to merely intended as an identifier to differentiate different features and does not require an order of operation unless otherwise specified. Accordingly, second cut 18 can be performed before first cut 12 without departing from the scope of the disclosure.

Forming wedge-shaped opening 10 can divide proximal phalanx 250 into a proximal portion 20 proximal of the wedge-shaped opening and a distal portion 22 distal of the wedge-shaped opening, with cut interface 11 separating proximal portion 20 of the proximal phalanx 250 from distal portion 22 of the proximal phalanx 250, at least along some of the interface therebetween. A small section of bone 24 may be retained between the apex of wedge-shaped opening 10 and the lateral-most side 16 of proximal phalanx 250. This section of bone 24 can act as a pivot or hinge about which distal portion 22 of proximal phalanx 250 is moved relative to proximal portion 20. Thus, the cut interface 11 can separate proximal portion 20 of the proximal phalanx 250 from distal portion 22 of the proximal phalanx 250 along some of the interface therebetween while the section of bone 24 can remain to connect proximal portion 20 of the proximal phalanx 250 to distal portion 22 of the proximal phalanx 250 along another, different portion of the interface therebetween. For example, after forming wedge-shaped opening 10, a clinician can move distal portion 22 of proximal phalanx 250 about the second of bone 24 in the transverse plane to close the wedge-shaped opening 10 by bringing the proximal portion 20 of the proximal phalanx 250 and distal portion 22 of the proximal phalanx 250 closer together. The clinician can move distal portion 22 of proximal phalanx 250 medially (thereby also moving distal phalanx 260 medially) relative to proximal portion 20 of proximal phalanx 250. Closing wedge-shaped opening 10 can bring a first cut end face 26 of proximal phalanx 250 in contact with a second cut end face 28 of the proximal phalanx. While the proximal portion 20 of the proximal phalanx 250 and distal portion 22 of the proximal phalanx 250 can be moved closer together to close the wedge-shaped opening 10 and bring the first cut end face 26 of proximal phalanx 250 in contact with the second cut end face 28 of proximal phalanx 250, though cut interface 11 can remain between the closed the first cut end face 26 of proximal phalanx 250 in contact with the second cut end face 28 of proximal phalanx 250.

One or more bone staples can then be applied, as will be described, across the closed osteotomy at the cut interface 11 to fixate the re-aligned position of the proximal portion 20 of the proximal phalanx 250 relative to the distal portion 22 of the proximal phalanx 250. This can include inserting at least one leg of the bone staple into proximal portion 20 of the proximal phalanx 250, inserting at least one leg of the bone staple into distal portion 22 of the proximal phalanx 250, and placing a bridge of the bone staple crossing the closed osteotomy (e.g., crossing the cut interface 11). In this way, the bone staple can then act to fixate the relative re-aligned position of the proximal portion 20 of the proximal phalanx 250 and the distal portion 22 of the proximal phalanx 250. As such, the bone staple can be so placed after performing an Akin osteotomy as a means to fixate the re-aligned bone position resulting from the Akin osteotomy.

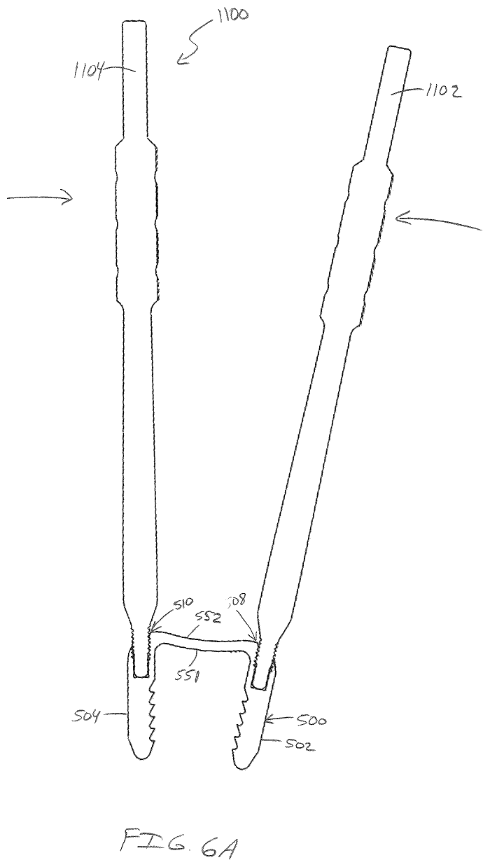

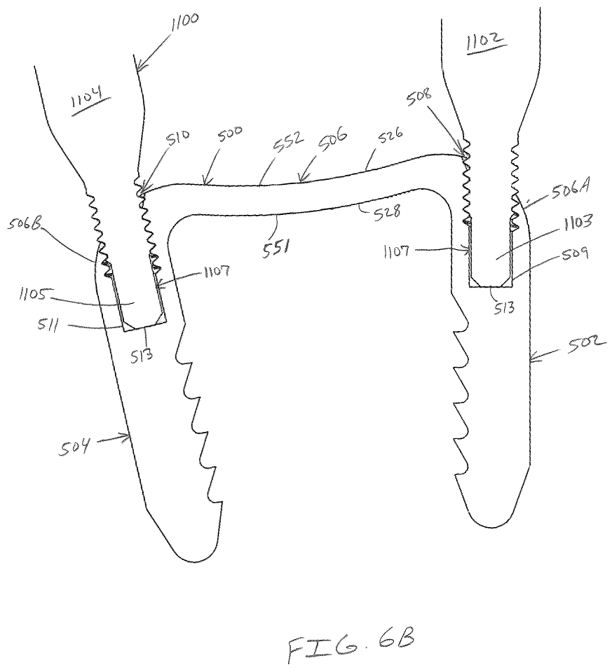

FIGS. 5A-5C show an embodiment of a bone staple 500. FIG. 5A is a perspective view of the bone staple 500, FIG. 5B is a top surface plan view of the bone staple 500, and FIG. 5C is an elevational view of the bone staple 500. The staple 500 can be configured to fixate two or more bone portions, for instance, such as after an Akin osteotomy. The staple 500 can include one or more features that configure the body of the staple 500 to anatomically fit a target implant region at each of the bone portions to be fixated relative to one another. In this way, the staple 500 can provide a more robust bone fixation by better conforming to the target bone surfaces where the staple 500 is to be positioned.

In the example disclosed here, staple 500 can conform to the anatomical bone surface contour at a metaphysis portion of the proximal phalanx. As such, the staple 500 can bridge across the cut interface between the re-aligned proximal portion of the proximal phalanx and distal portion of the proximal phalanx in a way that conforms to the anatomical bone surface contour at a metaphysis portion of the proximal portion and a metaphysis portion of the distal portion of the proximal phalanx. This conformance to the particular anatomical bone surface contour can enable more efficient transfer of fixation and/or stabilization forces from the staple 500 to the bone portions fixated relative to one another via the staple 500. This conformance to the particular anatomical bone surface contour can also help to reduce trauma to the anatomy, for instance by enabling smaller incision accesses and/or reducing agitation to the anatomy post-implant via the anatomical conformance of the staple 500. And, when the staple 500 is implanted, as described elsewhere herein, the staple 500 can be configured to apply a compression force at the bone potions and across the cut interface between the bone portions for use in fixating and/or promoting fusion of the bone portions.

The staple 500 can include a staple body 501 having a first leg 502, a second leg 504, and a bridge 506. For the illustrated embodiment, the staple 500 includes the first leg 502 at a first side 503 of the staple 500 and the second leg 504 at a second side 505 of the staple 500. In this example, the first side 503 is opposite the second side 505. The bridge 506 can connect the first leg 502 and the second leg 504.

The legs 502, 504 of the staple 500 can be configured for positioning in bones, such as one or more relatively small bones of the foot. This embodiment of the staple 500 can be configured to conform to a surface contour at a phalanx bone, such as to conform to a surface contour at each of a metaphysis portion of a first proximal phalanx bone portion and a metaphysis portion of a second proximal phalanx bone portion after an Akin osteotomy. Thus, the bridge 506 of the staple 500 can be configured to: (i) conform to a surface contour at a metaphysis portion of a first proximal phalanx bone portion, (ii) conform to a surface contour at a metaphysis portion of a second proximal phalanx bone portion, and (iii) extend across an interface (e.g., the cut interface after Akin osteotomy) between these first and second proximal phalanx bone portions (e.g., as re-aligned relative to one another as a result of the Akin osteotomy).

For example, a length 512 of each of the legs 502, 504 can be within a range from 4 mm to 30 mm, such as from 6 mm to 25 mm or from 8 mm to 20 mm, which can provide sufficient length to robustly anchor within a bone of the foot, such as a phalanx (e.g., first proximal phalanx). A width 514 of each of the legs can be within a range from 1 mm to 4 mm, such as from 1.5 mm to 3.5 mm or from 1.5 mm to 2.5 mm, which likewise can provide sufficient length to robustly anchor within a bone of the foot. A bridge length 516 of the bridge 506 can be within a range from 4 mm to 25 mm, such as from 6 mm to 20 mm, or from 8 mm to 12 mm (e.g., such as from 9-10 mm), which can be sufficient to allow for positioning the bridge across an interface (e.g., cut interface) between bones in the foot while maintaining the legs 502, 504 at such bones. A staple according to the disclosure can be configured with dimensions other than the foregoing examples, and the disclosure is not limited in this respect.

As an example shown at FIG. 5C, the bridge length 516 can be as measured from a central longitudinal axis of one leg 502 to a central longitudinal axis of another leg 504. As an example at FIG. 5B, the bridge 506 can also define a bridge width 570. In some examples, the bridge width 570 of the staple 500 is constant over the entire width of the staple. In other examples, the bridge width 570 of the staple 500 varies over the width of the staple. For example, the bridge width 570 can be substantially constant over a central region along the bridge length 516, and the bridge width 570 can increase moving along the bridge length 516 from the central region toward each of the first leg 502 and the second leg 504 (e.g., such that the greatest bridge width 570 is at or adjacent the first and second legs 502, 504). As one specific such example, the bridge width 570 can define a generally hourglass profile with the narrower, center of the hourglass at a central region along the bridge length 516 and the increasing width end portions of the hourglass at opposite ends of the bridge length 516.

As noted, the staple 500 can include one or more features that are configured to adapt the staple body 501 of the staple 500 to anatomically fit a target implant region at each of the bone portions to be fixated relative to one another. For the example illustrated here, the staple 500 can conform at the bridge 506 to the anatomical bone surface contour at a metaphysis portion of the proximal phalanx. As such, the bridge 506 can extend across the cut interface between the re-aligned proximal portion of the proximal phalanx and distal portion of the proximal phalanx in a way that conforms to the anatomical bone surface contour at a metaphysis portion of the proximal portion and a metaphysis portion of the distal portion of the proximal phalanx. This conformance of the bridge 506 to the particular anatomical bone surface contour at which the bridge 506 is to sit can enable more efficient transfer of fixation and/or stabilization forces from the staple 500 to the bone portions fixated relative to one another via the staple 500.

To enable the bridge 506 to conform to the anatomical bone surface contour at a metaphysis portion of the proximal phalanx, the bridge 506 can include a surface geometry at the bridge 506 that corresponds generally to the anatomical bone surface contour at a metaphysis portion of the proximal phalanx. For example, as shown here, the bridge 506 can include one or more concave and/or convex surfaces extending along some or all of the length 516 of the bridge 506. For instance, in some such examples, the bridge 506 can define a concave bridge (when viewed in a direction from the top side toward the bottom side) that moves downward toward legs 502, 504 moving in a direction from the respective leg 502, 504 along the bridge 506 to a central portion of the bridge 506. In particular, the bridge 506 can include a concave top surface 552 and a convex bottom surface 551 that extends along at least a portion of the bottom surface 528 of the staple 500 at the bridge 506. The convex bottom surface 551 can dip downward along the length of the bridge 506 in a same general direction (e.g., toward one or more bones) at which the legs 503, 504 extend out from the bridge 506. In some additional examples, such as that shown here, staple 500 can further include the concave top surface 552 extending along at least a portion of the top surface 526 of the staple 500 at the bridge 506. Likewise, the concave top surface 552 can dip downward along the length of the bridge 506 in a same general direction (e.g., toward one or more bones) at which the legs 503, 504 extend out from the bridge 506. In some such examples, such as that illustrated here, the convex bottom surface 551 and the concave top surface 552 at the bridge 506 can be symmetrical such that thickness 550 defined at bridge 506 can be constant between the concave and convex surfaces 551, 552 along at some or all of the length of the bridge 506.

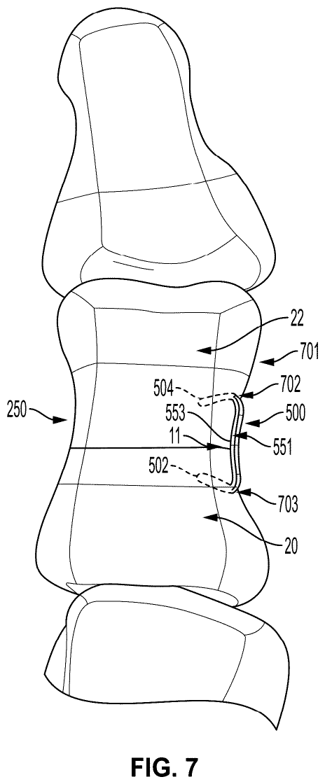

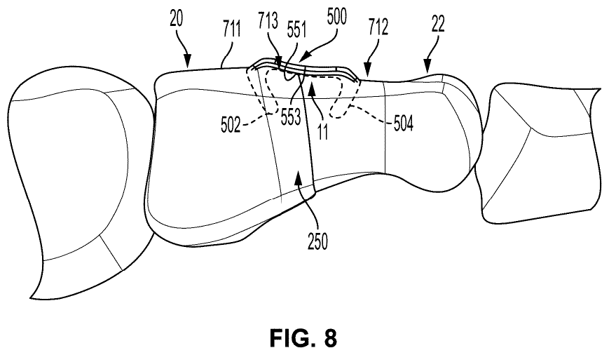

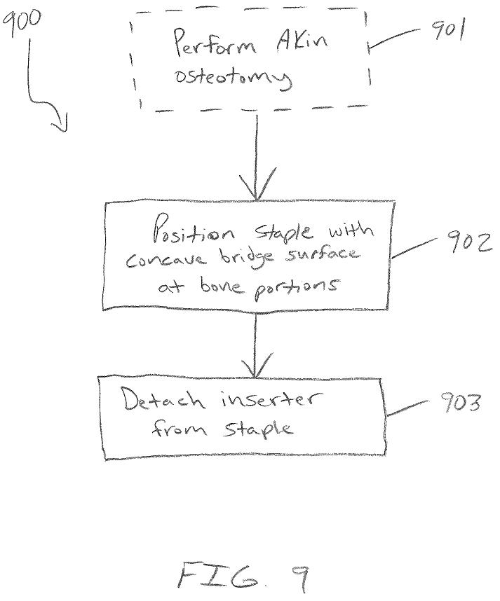

The convex bottom surface 551 at the bridge 506 can correspond to a curvature at a surface of a phalanx bone. For example, the convex bottom surface 551 at the bridge 506 can match a concave curvature at a surface of a metaphysis portion of the phalanx bone. Similarly, when the bridge 506 further includes the concave top surface 552, the concave top surface 552 can likewise match a concave curvature at a surface of a metaphysis portion of the phalanx bone. As illustrated, for instance, at FIG. 5C, the convex bottom surface 551 at the bridge 506 can define a local maximum point 553. This local maximum point 553 can define a lowest elevation of the bridge 506—an elevation of the bridge 506 closest to end portions of legs 504, 506 opposite bridge 506. The local maximum point 553 can be located at a central portion along length 516 of the bridge 506. In this way, the local maximum point 553 can be located along the length 516 of the bridge 506 so as to be spaced equally from each of the first handling coupling 508 and the second handling 510. Length 516 of bridge 506 between the first leg 502 and the second leg 504 can be configured to span across a cut interface at the surface of the metaphysis portion of the phalanx bone. Accordingly, as illustrated in more detail at the examples shown at FIGS. 7 and 8, the staple 500 can be configured to conform to natural contouring at target phalanx bone portion implant sites by enabling positioning of the staple 500 such that: (i) one side portion of the convex bottom surface 551 of bridge 506 is configured to sit at a surface of one phalanx bone portion, (ii) an opposite side portion of the convex bottom surface 551 of bridge 506 is configured to sit at a surface of another, interfacing phalanx bone portion, and (iii) the local maximum point 553 at the convex bottom surface 551 of bridge 506 is configured to sit at or near an interface between the phalanx bone portions (e.g., local maximum point 553 is configured to sit at the cut interface between the phalanx bone portions resulting from an Akin osteotomy).

The first leg 502 can include a first set of teeth 518 at a perimeter 522 of the first leg 502, and the second leg 504 can include a second set of teeth 520 at a perimeter 524 of the second leg 504. The teeth 518, 520 can extend out from the respective leg 502, 504 and be configured to provide an anchoring mechanism for maintaining the respective leg 502, 504 within the respective bone at which the respective leg 502, 504 is placed. As illustrated here, the first set of teeth 518 can extend partially around the perimeter 522 of the first leg 502, and the second set of teeth 520 can extend partially around the perimeter 524 of the second leg 504. For example, the first set of teeth 518 can extend around a portion of the perimeter 522 of the first leg 502 facing the bridge 506, and the second set of teeth 520 can extend around a portion of the perimeter 525 of the second leg 504 facing the bridge 506. As one specific such example, the first set of teeth 518 can extend around approximately one hundred and eighty degrees of the perimeter 522 of the first leg 502 nearest the bridge 506, and the second set of teeth 520 can extend around approximately one hundred and eighty degrees of the perimeter 524 of the second leg 504 nearest the bridge 506.

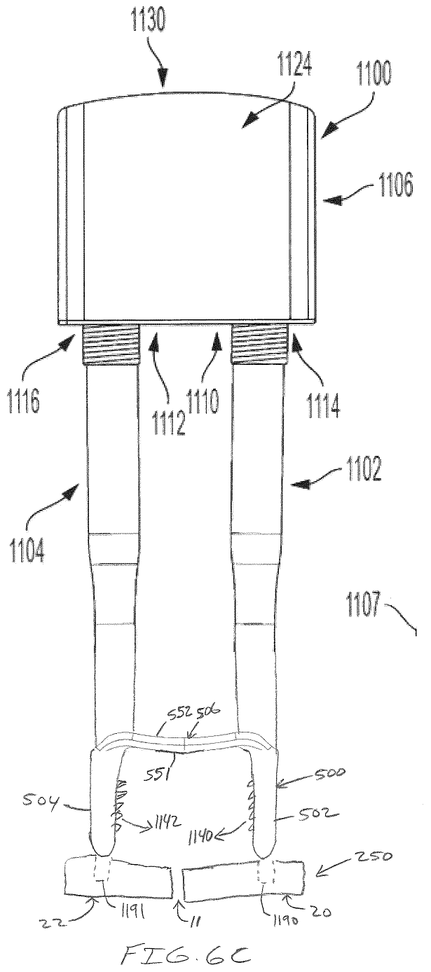

The staple 500 can further include a first handling coupling 508 and a second handling coupling 510 defined by the staple body 501. For the illustrated embodiment, the staple 500 includes the first handling coupling 508 at the first side 503 of the staple 500 and the second handling coupling 510 at the second side 505 of the staple 500. The first handling coupling 508 can include a first handling coupling receptacle 509 extending from a top surface 526 of the staple body 501 of the staple 500 toward (e.g., to) a bottom surface 528 of the staple body 501 of the staple 500. As one such specific example, the first handling coupling receptacle 509 can extend from the top surface 526 down a portion, but less than all of, the length 512 of the first leg 502 (e.g., as seen at FIG. 6B). The second handling coupling 510 can include a second handling coupling receptacle 511 extending from the top surface 526 of the staple body 501 of the staple 500 toward (e.g., to) the bottom surface 528 of the staple body 501 of the staple 500. As one such specific example, the second handling coupling receptacle 511 can extend from the top surface 526 down a portion, but less than all of, the length 512 of the second leg 504 (e.g., as seen at FIG. 6B). As such, the first handling coupling 508 and first handling coupling receptacle 509 as well as the second handling coupling 510 and second handling coupling receptacle 511 can be accessible from the top surface 526 of the staple 500, which can be useful in helping to facilitate generally flush placement of the bottom surface 528 of the staple 500 against one or more bones (e.g., against each of two bone portions separated by the cut interface resulting from the Akin osteotomy).

The first handling coupling receptacle 509 of the first handling coupling 508 can be configured to couple to a first coupling shaft of an inserter, such as at a location between the top surface 526 and a bottom surface 513 (shown, e.g., at FIG. 6B) of the handling coupling receptacle. The second handling coupling receptacle 511 of the second handling coupling 510 can be configured to couple to a second coupling shaft of an inserter, such as at a location between the top surface 526 and a bottom surface 513 (shown, e.g., at FIG. 6B) of the handling coupling receptacle. As such, the first and second handling coupling receptacles 509, 511 can be configured to operatively couple to the respective first and second coupling shafts of the inserter such that the first and second coupling shafts of the inserter are inserted into the respective first and second handling coupling receptacles 509, 511 from the top surface 526 and maintained within the respective first and second handling coupling receptacles 509, 511 so as to not extend out from the bottom surface of the handling coupling receptacles. The bottom end of each handling coupling receptacle can be closed with a solid portion first leg 502 or second let 504, respectively, extending beyond the bottom end of the handling coupling receptacle.

In the illustrated example, the first and second handling coupling receptacles 509, 511 includes threads extending along a length of the first and second handling coupling receptacles 509, 511 between the top and bottom surfaces of the handling coupling receptacle. These threads can be configured to connect to complementary threads on respective first and second coupling shafts of an inserter. Though in other embodiments the first and second handling coupling receptacles 509, 511 and first and second coupling shafts of the inserter can include others means to facilitate an operative connection therebetween the respective components (e.g., a bayonet connection).

Depending on the application in which the staple 500 is used, the staple 500 can be configured to receive one or more solid or liquid substances after insertion of the staple into bone. As one such example, one or both of the first and second handling coupling receptacles 509, 511 can be configured to receive a filler material therein to substantially plug the first and/or second handling coupling receptacles 509, 511 at the top surface 526. This filler material can be placed in the first and/or second handling coupling receptacles 509, 511 after removing the respective first and/or second coupling shaft from the respective first and/or second handling coupling receptacles 509, 511. For instance, a biologically compatible wax or other biologically compatible filler material can be placed into the first and/or second handling coupling receptacles 509, 511 to plug the first and/or second handling coupling receptacles 509, 511 at or near the top surface 526 so as to help impede bone ingrowth and/or passage of biologic substances into the first and/or second handling coupling receptacles 509, 511 after removing corresponding inserter coupling shafts.

Additionally or alternatively, the staple 500 can be configured with a cannulation extending through the length of leg 502 and/or 504 for receiving corresponding wires inserted into bone to help facilitate positioning and placement of the staple into underlying bone. For example, in lieu of using an inserter having wire receiving openings to guide positioning of an implant as will be described, wires inserted into underlying bones can be aligned with cannulations extending through at least two legs of the staple. The cannulations can be aligned with the wires positioned in the bones and the staple guided along the wires.

The first leg 502 can define, and length 512 of the leg can extend along, a first leg central longitudinal axis 530 extending through a geometric center of the leg 502. The second leg 504 can define, and the length 512 of the leg can extend along, a second leg central longitudinal axis 532 extending through a geometric center of the leg 504. Likewise, the first handling coupling receptacle 509 can define and extend a length from the top surface 526 toward (e.g., to) a bottom surface along a first handling coupling receptacle central longitudinal axis 534 that extends through a geometric center of the handling coupling receptacle. The second handling coupling receptacle 511 can define and extend a length from the top surface 526 toward (e.g., to) a bottom surface along a second handling coupling receptacle central longitudinal axis 536 that extends through a geometric center of the handling coupling receptacle. As shown for the illustrated embodiment of the staple 500, the first leg central longitudinal axis 530 can be co-axial with the first handling coupling receptacle central longitudinal axis 534, and the second leg central longitudinal axis 532 can be co-axial with the second handling coupling receptacle central longitudinal axis 536.

The staple 500 can have a thickness 550 that can differ at different regions of the staple 500. For example, the staple 500 can have a bridge thickness 550a at the bridge 506 and a thickness transition region 550b where the bridge 506 transitions to the respective first leg 502 and the second leg 504. As shown for the illustrated embodiment, the thickness transition region 550b can be greater than the bridge thickness 550a (e.g., at a central location of the bridge along the bridge length 516). In particular, the thickness transition region 550b can include an increase in thickness of the staple 500 moving in a direction from the bridge 506 toward the respective leg 502, 504. In one example, the first handling coupling 508 and the first handling coupling receptacle 509 can be located at the thickness transition region 550b adjacent the first leg 502, and the second handling coupling 510 and the second handling coupling receptacle 511 can be located at the thickness transition region 550b adjacent the second leg 504. Such location of the first handling coupling 508 and the first handling coupling receptacle 509 as well as the second handling coupling 510 and the second handling coupling receptacle 511 at the increased thickness portion of the staple 500 can help to increase the strength of the staple 500 for receiving a load force.

As noted, the staple 500 can be configured to have a natural, undeformed state, an example of such state is shown at FIGS. 5A-5C. The staple 500 can be configured to transition to a deformed insertion state upon application of a load force at the staple 500. The staple 500 can have a biased, compression-inducing state where the first leg 502 and the second leg 504 are angled toward one another, which can help to apply a compression force to urge the bones in which the staple 500 is positioned together, applying a compression force across the separation (e.g., cut interface resulting from Akin osteotomy) between the bones such that the end faces of the opposed bones are pressed together.

Upon application of a load force to the staple 500, the staple 500 can be configured to transition from a undeformed state in which the legs of the staple are at their natural or resting positions to a deformed insertion state at which the first and second legs 502, 504 (e.g., end portion 537 of first leg 502 and end portion 538 of second leg 504) are spaced further apart (e.g., and oriented generally parallel to one another) as compared to the natural state. In particular, the staple 500 can be configured such that upon application of the load force at the staple 500, the end portion 537 of first leg 502 is configured to move in a direction 540 (e.g., away from the bridge 506) and the end portion 538 of second leg 504 is configured to move in a direction 542 (e.g., away from the bridge 506) from the undeformed state to the deformed insertion state. Conversely, upon reduction or removal of the applied load force at the staple 500, the staple 500 can be configured such that the end portion 537 of first leg 502 is configured to move in a direction opposite the direction 540 (e.g., toward the bridge 506) from the deformed insertion state back toward the undeformed state, and the end portion 538 of second leg 504 is configured to move in a direction opposite the direction 542 (e.g., toward the bridge 506) from the deformed insertion state back toward the deformed state.

In use, the staple 500 can provide compression across the end faces of the bone portions into which the staple 500 is inserted. Compression can occur when the legs of the staple are inserted into the bones (e.g., into pre-drilled openings in the bones) at a spacing and/or angle greater than the natural, undeformed configuration of the legs. The staple legs can be deformed to be inserted into the bones and, when the force applied to deform the legs is released, the staple legs can elastically bias toward their unbiased (natural or undeformed) shape. However, the spacing and/or angulation of the legs inserted into the bones can prevent the legs from fully returning to their undeformed state. As a result, the staple can apply a compressive force between the end faces of the bones into which the staple legs are inserted (e.g., with the force directed in the direction of convergence of the staple legs). The compressive force may help promote bone healing and fusion between the bones into which the staple is inserted.

The staple 500 can be configured to maintain one or more of the features for conforming to the anatomical bone surface contour (e.g., at a metaphysis portion of the proximal phalanx) both in the undeformed state of the staple 500 without load force applied and in the deformed state of the staple 500 with load force applied. For example, the bridge 506 can be configured to maintain surface geometry at the bridge 506 that corresponds generally to the anatomical bone surface contouring (e.g., metaphysis portions of the proximal phalanx bone portions) where the staple 500 is to be placed. As one such example, the bridge 506 can be configured to maintain the presence of the convex bottom surface 551 (e.g., whether to the same degree of convexity or to a lesser degree of convexity along the convex bottom surface 551) at the bridge 506 both when the load force is applied to the staple and when the load force is removed from the staple 506. When the staple 500 also includes the concave top surface 552, the staple 500 can be configured such that each of the convex bottom surface 551 at the bridge 506 and the concave top surface 552 at the bridge 506 is present both when the load force is applied and when the load force is removed.

FIGS. 6A-6C show an embodiment of an inserter 1100 coupled to the staple 500. For example, the inserter 110 can be used to apply the load force to the staple 500. FIG. 6A is a cross-sectional view of the inserter 1100 coupled to the staple 500, FIG. 6B is a close-up view of the cross-section at FIG. 6A showing the inserter 1100 coupled to the staple 500 via handling couplings 508, 510 at the staple 500, and FIG. 6C is an elevational view of the inserter 1100 coupled to the staple 500 to apply a load force to the staple 500 and to position the staple 500 across bone portions (e.g., across phalanx bone portions interfacing a cut interface after an Akin osteotomy).

The inserter 1100 can include a first coupling shaft 1102, a second coupling shaft 1104, and a connector 1106. The first and second coupling shafts 1102, 1104 can be configured to operatively connect to staple 500. The connector 1106 can be configured to join the first coupling shaft 1102 and the second coupling shaft 1104, for instance as shown at the example at FIG. 6C.

In particular, the first coupling shaft 1102 can be configured to operatively couple to the staple 500 at the first handling coupling 508, and the second coupling shaft 1104 can be configured to operatively couple to the second handling coupling 510. The inserter 1100 can include the first coupling shaft 1102 connected to a first side of the implant, such as the first side 503 of the staple 500, and the second coupling shaft 1104 connected to a second side of the implant, such as the second side 505 of the staple 500. For example, the first coupling shaft 1102 can have a distal end portion 1103 and the second coupling shaft 1104 can have a distal end portion 1105, and each of such distal end portions 1103, 1105 can include an implant coupling member 1107. The coupling member 1107 at the distal end portion 1103 of the first coupling shaft 1102 can be configured to operatively connect to a complementary coupling member at the first handling coupling 508, and the coupling member 1107 at the distal end portion 1105 of the second coupling shaft 1104 can be configured to operatively connect to a complementary coupling member at the second handling coupling 510. As shown for the example here, the coupling member 1107 can define a tapered nose at the respective distal end portion 1103, 1105 that tapers in width moving toward the terminal end of the respective distal end portion 1103, 1105. This tapered nose geometry at the inserter 1100 can be useful in enabling the transfer of load force from the inserter 1100 to the staple 500 in a way that helps to maintain the concave/convex curvature at the bridge 506 (e.g., maintain the convex bottom surface 551 and/or the concave top surface 552 at the bridge 506).

As one such specific example, each of the first and second handling couplings 508, 510 can include threading as a type of complementary coupling member thereat. The coupling member 1107 at the distal end portions 1103, 1105 of the respective first and second coupling shafts 1102, 1104 can include threading that is configured to operatively couple to the complementary threading at the respective first and second handling couplings 508, 510. Thus, in this particular example, operatively coupling the inserter 1100 to the staple 500 can include threadingly inserting the first coupling shaft 1102 into the first handling coupling 508 (e.g., from the top surface 526 of the staple 500) and threadingly inserting the second coupling shaft 1104 into the second handling coupling 510 (e.g., from the top surface 526 of the staple 500). Other types of mechanical connections than threading can be used.

As seen at FIG. 6B, each of the first coupling shaft 1102 and the second coupling shaft 1104 can be configured to couple to the respective first and second handling coupling receptacles 509, 511 at a location between the top surface 526 of the staple 500 and the bottom surface 528 of the staple 500. For example, the first coupling shaft 1102 can operatively couple to the first handling coupling 508 from the top surface 526 and in a direction toward the bottom surface 528 but without the first coupling shaft 1102 extending out from the bottom surface 528. Likewise, the second coupling shaft 1104 can operatively couple to the second handling coupling 510 from the top surface 526 and in a direction toward the bottom surface 528 but without the second coupling shaft 1104 extending out from the bottom surface 528. As one such example, the coupling member 1107 at the first coupling shaft 1102 can extend within the first handling coupling receptacle 509 such that a distal end of the coupling member 1107 at the first coupling shaft 1102 is contained within the staple 500 (e.g., within the first handling coupling receptacle 509 and within the leg 502). Similarly, the coupling member 1107 at the second coupling shaft 1104 can extend within the second handling coupling receptacle 511 such that a distal end of the coupling member 1107 at the second coupling shaft 1104 is contained within the staple 500 (e.g., within the second handling coupling receptacle 511 and within the leg 504).

The inserter 1100 can be configured to couple to the staple 500 through the top surface 526 of the staple 500. The first handling coupling 508 can extend through the top surface 526 of the staple 500 on the first side 503 of the staple 500, and the second handling coupling 510 can extend through the top surface 526 of the staple 500 on the second side 505 of the staple 500. The inserter 1100 can be configured to couple to the first handling coupling 508 through the top surface 526 of the staple 500 without extending under the bottom surface 528 of the staple 500, and the inserter 1100 can be configured to couple to the second handling coupling 510 through the top surface 526 of the staple 500 without extending under the bottom surface 528 of the staple 500. In one exemplary application where the staple 500 is to be placed at phalanx bone portions and bridging across an interface between those phalanx bone portions (e.g., cut interface resulting from Akin osteotomy), when the inserter 1100 is connected to the first handling coupling 508 and/or the second handling coupling 510 through the top surface 526 without extending under the bottom surface 528 of the staple 500, the bottom surface 528, such as the convex bottom surface 551 along the bridge 506, can be configured to directly contact the a surface of the interfacing phalanx bone portions without any inserter 1100 structure present between the bottom surface 528 and the surfaces of the interfacing phalanx bone portions where the staple 500 is to sit. This configuration can allow the convex bottom surface 551 of the staple 500 to be placed more flushly at the surfaces of the interfacing phalanx bone portions as compared to a configuration where an inserter structure is present between the convex bottom surface 551 and the phalanx bone portion surface when such inserter is coupled to the staple 500. With the inserter 1100 coupled to the first and second handling couplings 508, 510 through the top surface 526 of the staple 500 without extending under the bottom surface 528 of the staple 500, the inserter 1100 can be configured to apply the load force at the staple 500 to cause the first second legs 502, 504 to move apart from one another.

In some additional or alternative examples where the inserter 1100 is configured to couple to the staple 500 through the top surface 526 of the staple 500, the inserter 1100 can be configured to connect to the first side 503 of the staple 500 through the top surface 526 without extending under the bottom surface 528 and without contacting an outer perimeter of the first side 503 of the staple 500, and the inserter 1100 can be configured to connect to the second side 505 of the staple 500 through the top surface 526 without extending under the bottom surface 528 and without contacting an outer perimeter of the second side 505 of the staple 500. For example, as shown at FIG. 6B, the inserter 1100 can be configured to connect to the first side 503 of the staple 500 through the top surface 526 without extending under the bottom surface 528 and without contacting an outer perimeter of the first side 503 of the staple 500 formed by the sidewall 506A which connects the top surface 526 of the staple 500 to the bottom surface 528 of the staple 500, and, similarly, the inserter 1100 can be configured to connect to the second side 505 of the staple 500 through the top surface 526 without extending under the bottom surface 528 and without contacting an outer perimeter of the second side 505 of the staple 500 formed by the sidewall 506B.

In some additional or alternative examples where the inserter 1100 is configured to couple to the staple 500 through the top surface 526 of the staple 500, the staple 500 and the inserter 1100 can be configured such that, when the inserter 1100 is coupled to the first handling coupling 508, the inserter 1100 contacts first handling coupling 508 and the inserter 1100 is isolated at the first handling coupling 508 from any contact with a perimeter sidewall (e.g., sidewall 506A) of the staple 500 connecting the top surface 526 of the staple 500 to the bottom surface 528 of the staple 500. Similarly, the staple 500 and inserter 1100 can further be configured such that, when the inserter 1100 is coupled to the second handling coupling 510, the inserter 1100 contacts the second handling coupling 510 and the inserter 1100 is isolated at the second handling coupling 510 from any contact with the perimeter sidewall (e.g., sidewall 506B) of the staple 500. As one particular such example, the staple 500 and the inserter 1100 can be configured such that, when the inserter 1100 is coupled to the staple 500 at the first handling coupling 508, the inserter 1100 is confined to contacting the staple 500 at only the first handling coupling receptacle 509 defined by the first handling coupling 508, and, when the inserter 1100 is coupled to the staple 500 at the second handling coupling 510, the inserter 1100 is confined to contacting the staple 500 at only the second handling coupling receptacle 511 defined by the second handling coupling 510.

In various embodiments, the inserter 1100 (e.g., coupling shafts 1102, 1104) can be configured to couple to the staple 500 through the top surface 526 of the staple 500 such that only the staple 500, and no portion of the inserter 1100 (e.g., no portion of the coupling shafts 1102, 1104), defines a contact interface with the one or more bones at which the staple 500 is being inserted into. This coupled configuration of the staple 500 and inserter 1100 (e.g., coupling shafts 1102, 1104) that results in staple only contact at the one or more bones —including convex bottom surface 551 at bridge 506—at which the staple 500 is being inserted into can help to insert the staple 500 flushly at the one or more bones because no inserter 1100 structure (e.g., no coupling shaft 1102, 1104 structure) is present at a location to contact the one or more bones which would prevent flush insertion of the staple 500 at the one or more bones.

In some examples where the inserter 1100 is configured to couple to the staple 500 through the top surface 526 of the staple 500 and includes one or more shaft stabilizing arm(s), the inserter 1100 can further be configured to couple to the first handling coupling 508 through the top surface 526 of the staple 500 without extending down an entire thickness 550 of a perimeter sidewall (e.g., sidewall 506A) of the staple 500 connecting the top surface 526 of the staple 500 to the bottom surface 528 of the staple 500. Similarly, the inserter 1100 can be further configured to couple to the second handling coupling 510 through the top surface 526 of the staple 500 without extending down the entire thickness 550 of the perimeter sidewall (e.g., sidewall 506B) of the staple 500 connecting the top surface 526 of the staple 500 to the bottom surface 528 of the staple 500. In an alternate example where the inserter 1100 is configured to couple to the staple 500 through the top surface 526 of the staple 500, the inserter 1100 can further be configured to: (i) couple to the first handling coupling 508 through the top surface 526 of the staple 500 without extending down more than three-quarters of the perimeter sidewall (e.g., sidewall 506A) of the staple 500 connecting the top surface 526 of the staple 500 to the bottom surface 528 of the staple 500, and (ii) couple to the second handling coupling 510 through the top surface 526 of the staple 500 without extending down more than three-quarters of the perimeter sidewall (e.g., sidewall 506A) of the staple 500 connecting the top surface 526 of the staple 500 to the bottom surface 528 of the staple 500. In another alternate example where the inserter 1100 is configured to couple to the staple 500 through the top surface 526 of the staple 500, the inserter 1100 can further be configured to: (i) couple to the first handling coupling 508 through the top surface 526 of the staple 500 without extending down more than half of the perimeter sidewall (e.g., sidewall 506A) of the staple 500 connecting the top surface 526 of the staple 500 to the bottom surface 528 of the staple 500, and (ii) couple to the second handling coupling 510 through the top surface 526 of the staple 500 without extending down more than half of the perimeter sidewall (e.g., sidewall 506A) of the staple 500 connecting the top surface 526 of the staple 500 to the bottom surface 528 of the staple 500. In these noted examples, the thickness 550 of the perimeter sidewall (e.g., sidewall 506A) of the staple 500 connecting the top surface 526 of the staple 500 to the bottom surface 528 of the staple 500 can be at least 0.5 mm. As such, in the example where the inserter 1100 is configured to couple to the first and second handling couplings 508, 510 without extending down more than three-quarters of the perimeter sidewall (e.g., sidewall 506A) of the staple 500, the inserter can be configured to so couple without extending down more than three-quarters of the of the at least 0.5 mm thickness 550 at the perimeter sidewall (e.g., sidewall 506A) of the staple 500. And in the example where the inserter 1100 is configured to couple to the first and second handling couplings 508, 510 without extending down more than half of the perimeter sidewall (e.g., sidewall 506A) of the staple 500, the inserter can be configured to so couple without extending down more than half of the of the at least 0.5 mm thickness 550 at the perimeter sidewall (e.g., sidewall 506B) of the staple 500.

The inserter 1100 can further include a first wire receiving opening 1110, a second wire receiving opening 1112, a first receptacle 1114, and a second receptacle 1116. The first wire receiving opening 1110 can be configured to receive a first wire, and the second wire receiving opening 1112 can be configured to receive a second wire. The first receptacle 1114 can be configured to receive and hold the first coupling shaft 1102, for instance a proximal end portion 1120 of the first coupling shaft 1102, and the second receptacle 1116 can be configured to receive and hold the second coupling shaft 1104, for instance a proximal end portion 1122 of the second coupling shaft 1104. For the illustrated embodiment, each of the first wire receiving opening 1110, the second wire receiving opening 1112, the first receptacle 1114, and the second receptacle 1116 is included at the connector 1106.

As noted, the connector 1106 can be configured to join the first coupling shaft 1102 and the second coupling shaft 1104, for instance as shown at the example of FIG. 6C. As one example, the connector 1106 can be configured to join the first and second coupling shafts 1102, 1104 by receiving the first coupling shaft 1102 at the first receptacle 1114 at the connector 1106 and receiving the second coupling shaft 1104 at the second receptacle 1116 at the connector 1106. As such, with the receptacles 1114, 1116 at the connector 1106 receiving and holding the respective coupling shaft 1102, 1104, the connector 1106 can removably join the coupling shafts 1102, 1104.

As one specific such example, the connector 1106 as shown for the illustrated embodiment of the inserter 1100 can include a cap 1124. For the illustrated embodiment of the inserter 1100, the cap 1124 can be configured to be positioned over the proximal end portion 1120 (e.g., opposite the implant, such as the staple 500) of the first coupling shaft 1102 and over the proximal end portion 1122 (e.g., opposite the implant, such as the staple 500) of the second coupling shaft 1104. For instance, the cap 1124 can be configured to join the first and second coupling shafts 1102, 1104 by placing the first receptacle 1114 at the cap 1124 over the proximal end portion 1120 of the first coupling shaft 1102 and the second receptacle 1116 at the cap 1124 over the proximal end portion 1122 of the second coupling shaft 1104, and then moving the cap 1124 as so positioned relative to the first and second coupling shafts 1102, 1104 (e.g., in a direction toward one or more bones) such that the proximal end portions 1120, 1122 are received and held at the respective receptacles 1114, 1116. Likewise, the cap 1124 can similarly be configured to receive and hold first and second wires, positioned at one or more bones, at respective first and second wire receiving openings 1110, 1112 as the cap 1124 is moved relative to such wires (e.g., in a direction toward one or more bones).

The cap 1124 can further include a surface contour 1130. The surface contour 1130 can be adapted to fit at a hand of a user. For the illustrated example, the surface contour 1130 can have a highest elevation at a location between the receptacles 1114, 1116 and a lowest elevation outside of the receptacles 1114, 1116 such that the surface contour 1130 angles downward toward the implant, such as the staple 500, when moving along the surface contour 1130 away from the highest elevation between the receptacles 1114, 1116. Accordingly, when positioning the implant, such as the staple 500, in contact with the first bone and the second bone, a user's hand can tamp at the surface contour 1130 of the cap 1124 to apply insertion force at the implant, such as the staple 500.

The inserter 1100 can be configured to place the implant, such as the staple 500, in one or more bones. For example, the inserter 1100 can be operatively connected to the staple 500 via the first and second coupling shafts 1102, 1104, and the connector 1106 (e.g., cap 1124) can be joined to the first and second coupling shafts 1102, 1104, such as shown at the example of FIG. 6C. When the connector 1106 is joined to the first coupling shaft 1102 and the second coupling shaft 1104, such as shown at the example of FIG. 6C, the connector 1106 can be configured to bias the first coupling shaft 1102 and the second coupling shaft 1104 toward each other to apply a load force to the staple 500. When the first coupling shaft 1102 and the second coupling shaft 1104 are so biased toward each other to apply the load force to the staple 500, the first leg 502 and the second leg 504 can be oriented generally parallel to one another, such as shown at the example of FIG. 6C. This application of the load force to the staple 500 can cause the legs 502, 504 to move away from each other to the generally parallel orientation which can be a configuration useful for inserting the staple 500 in proximal portion 20 of the proximal phalanx and distal portion 22 of the proximal phalanx. Then, after the staple 500 has been inserted as desired at and across the bone portions 20, 22, the connector 1106 can be removed from at least one of the first coupling shaft 1102 and the second coupling shaft 1104 to cause the first leg 502 and the second leg 504 to move toward one another. Specifically, removing the connector 1106 can cause the first leg 502 to move in a direction 1140 toward the second leg 504 and cause the second leg 504 to move in a direction 1142 toward the first leg 502.

Thus, upon application of a load force via the inserter 1100, the staple 500 can be configured to transition between an undeformed state, such as shown at FIG. 6A, where the legs 502, 504 point toward one another, to a deformed state, such as shown at FIG. 6C, where the legs 502, 504 are generally parallel to one another. For example, the load force can be applied at the staple 500 to transition the staple 500 to the deformed state for inserting the legs 502, 504 into respective holes 1190, 1191 at respective proximal and distal portions 20, 22 of the proximal phalanx, with the bridge 506 maintaining the presence of the convex bottom surface 551. Then, when the legs 502, 504 have been positioned at the respective proximal and distal portions 20, 22 of the proximal phalanx and with the convex bottom surface 551 bridging across the cut interface 11, the load force can be removed from the staple 500 (e.g., by removing the cap 1106 from the coupling shafts 1102, 1104) to cause the staple 500 to transition back to the undeformed state where the legs 502, 504 point toward one another to cause the legs 502, 504 to apply compressive force at the proximal and distal portions 20, 22 of the proximal phalanx to stabilize and fixate the bone portions 20, 22 across the cut interface 11. As described previosuly herein, the staple 500 can maintain the convex bottom surface 551 at the bridge in both the deformed and undeformed states such that the bridge 506 can be configured to conform to the anatomic contour at each of the bone portions 20, 22 in both the deformed and undeformed states. This can be useful in increasing the compressive force transfer while helping to reduce incision site size and/or trauma to the localized implant region.

FIG. 7 is a plan view showing the staple 500 positioned at a medial side 701 of, and across cut interface 11 at, a phalanx bone 250 (e.g., first proximal phalanx). As shown here, the staple 500 includes a geometric profile that can conform to curvature at a surface at the medial side 701 of phalanx bone 250. Namely, as shown here, the medial side 701 of phalanx bone 250 can include at bone portion 20 concave curvature extending inward toward bone 250 from the medial side 701 at medial metaphysis portion 703 and can include at bone portion 22 concave curvature extending inward toward bone 250 from the medial side 701 at medial metaphysis portion 702. The convex bottom surface 551 at the bridge 506 of the staple 500 can: (i) conform to the concave surface contour at medial metaphysis portion 703 of first proximal phalanx bone portion 20, (ii) conform to the concave surface contour at medial metaphysis portion 702 of a second proximal phalanx bone portion 22, and (iii) extend across cut interface 11 (e.g., resulting from an Akin osteotomy) between these first and second proximal phalanx bone portions 20, 22 (e.g., as re-aligned relative to one another as a result of the Akin osteotomy). For instance, as shown here, the local maximum point 553 along the convex bottom surface 551 at the bridge 506 of the staple 500 can be aligned generally with the cut interface 11 such that the convex bottom surface 551 extending to each side of the local maximum point 553 can be positioned at the respective medial metaphysis portions 702, 703.

FIG. 8 is an elevational view showing the staple 500 positioned at a dorsal side 711 of, and across a cut interface 11 at, a phalanx bone 250 (e.g., first proximal phalanx). As shown here, the staple 500 includes a geometric profile that can conform to curvature at a surface at the dorsal side 711 of phalanx bone 250. Namely, as shown here, the dorsal side 711 of phalanx bone 250 can include at bone portion 20 concave curvature extending inward toward bone 250 from the dorsal side 711 at dorsal metaphysis portion 713 and can include at bone portion 22 concave curvature extending inward toward bone 250 from the dorsal side 711 at dorsal metaphysis portion 712. The convex bottom surface 551 at the bridge 506 of the staple 500 can: (i) conform to the concave surface contour at dorsal metaphysis portion 713 of first proximal phalanx bone portion 20, (ii) conform to the concave surface contour at dorsal metaphysis portion 712 of a second proximal phalanx bone portion 22, and (iii) extend across cut interface 11 (e.g., resulting from an Akin osteotomy) between these first and second proximal phalanx bone portions 20, 22 (e.g., as re-aligned relative to one another as a result of the Akin osteotomy). For instance, as shown here, the local maximum point 553 along the convex bottom surface 551 at the bridge 506 of the staple 500 can be aligned generally with the cut interface 11 such that the convex bottom surface 551 extending to each side of the local maximum point 553 can be positioned at the respective dorsal metaphysis portions 712, 713.

FIG. 9 is a flow diagram of an embodiment of a method 900 for fixating one or more bones. The method 900 can be executed, for instance, using the staple 500 and/or inserter 1100 having one or more (e.g., each) of the features as disclosed elsewhere herein.

As one exemplary application, the method 900 can be used in conjunction with an Akin osteotomy. In this exemplary application, the method 900 can include an initial step at 901 of performing an Akin osteotomy, for instance, at proximal phalanx bone (e.g., first proximal phalanx bone in the foot). When included, step 910 can include realigning bone portions relative to one another using a cut interface made between the bone potions to be realigned to impart this relative movement. When the cut interface is made and the bone portions have been relatively realigned as desired, the method 900 can then proceed to include one or more steps relating to fixating this realigned bone positioning.

At step 902, the method 900 can include positioning a staple with a concave and/or convex bridge surface at first and second bone portions (e.g., first and second bone realigned portions resulting from the Akin osteotomy). When the method 900 includes the step 901, the step 902 of positioning the staple can occur after performing the Akin osteotomy at the first bone portion and the second bone portion. For example, this can include positioning a first leg of the staple connected to an inserter in a first implant hole of the first bone portion and a second leg of the staple connected to the inserter in a second implant hole of the second bone portion. The inserter can be connected to the staple through the top surface of the staple (e.g., opposite the convex bottom bridge surface). The first leg of the staple can be connected to the second leg of the staple by the bridge, the staple can be positioned at step 902 such that a bottom surface of the staple faces the first bone portion and the second bone portion and a top surface of the staple faces away from the first bone portion and the second bone portion. The bridge of the staple includes a convex bottom surface as a convex bridge surface extending along at least a portion of the bottom surface of the staple at the bridge, and the staple can be positioned at step 902 with the convex bottom bridge surface at each of the first bone portion and the second bone portion.

As one example at step 902, the staple can be positioned with the convex bottom surface at each of a concave curvature at a surface of a metaphysis portion of the first bone portion and a concave curvature at a surface of a metaphysis portion of the second bone portion. For instance, the first bone portion can be a first portion of a phalanx bone and the second bone portion can be second portion of the phalanx bone, with the first portion of the phalanx bone separated from the second portion of the phalanx bone by a cut interface or other interface between these bone portions. This can include the staple being positioned such that the convex bottom surface at the bridge extends from the first portion of the phalanx bone to the second portion of the phalanx bone and across the cut interface or other interface between these bone portions. In some such instances, the convex bottom surface at the bridge can define a local maximum point, and the staple can be positioned such that the local maximum point of the convex bottom surface at the bridge is at least the cut interface.

For some applications of the method 900, additional steps can be included between the positioning step 902 and the detaching step 903.

For example, the method 900 can include a step of applying a load force at the staple using the inserter, and, as the load force is applied at the staple using the inserter, the first leg and the second leg are caused to move apart from one another. Additionally, the method 900 can include another, subsequent step of removing the load force from the staple (i) after positioning the first leg in the first implant hole and the second leg in the second implant hole at step 902, (ii) after positioning the convex bottom surface at the bridge at each of the first bone portion and the second bone portion at step 902, and (iii) prior to detaching the inserter from the staple at step 903. For instance, removing the load force from the staple can cause the first leg and the second leg to move toward one another to apply a compression force at the first bone and the second bone while maintaining the convex bottom surface at each of the first bone portion and the second bone portion. In one such instance, the inserter can include a first coupling shaft connected through the top surface of the staple at the first leg and a second coupling shaft connected through the top surface of the staple at the second leg, and applying the load force can include biasing the first coupling shaft and the second coupling shaft toward each other. In some such applications, a connector can join the first coupling shaft and the second coupling shaft to bias the first coupling shaft and the second coupling shaft toward each other and maintain the load force at the staple while the connector joins the first coupling shaft and the second coupling shaft. When a connector is so used, an additional step in the method 900 can then include removing the connector from the first coupling shaft and the second coupling shaft to cause the first coupling shaft to move away from the second coupling shaft while maintaining the convex bottom surface at the bridge of the staple.

At step 903, the method 900 can include detaching the inserter from the staple. For example, the inserter can be removed from one or more handling couplings at the staple once the staple has been sufficiently placed to impart the compressive force at the bone portions. Detaching the inserter from the staple at step 903 can include maintaining the convexity at the convex bottom surface of the bridge to enable the staple to conform to the surface contour at the bone portions where the staple is placed.

Additional features, devices, and techniques can be utilized in conjunction with the present disclosure. As one example, the features, devices, and techniques for implanting a staple as disclosed in U.S. Pat. No. 18/467,686, the entire of contents of which are hereby incorporated by reference.

Various examples have been described. These and other examples are within the scope of the following claims.

1. A method of performing an Akin osteotomy, the method comprising:
cutting a wedge-shaped opening into a proximal phalanx of a foot, the wedge-shaped opening extending from a medial side of the proximal phalanx toward a lateral side of the proximal phalanx and dividing the proximal phalanx into a first bone portion and a second bone portion joined by a bridge of bone on a lateral side of the proximal phalanx;
moving the first bone portion and the second bone portion relative to each other to close the wedge-shaped opening, thereby bringing a cut face of the first bone portion in contact with a cut face of the second bone portion;
fixating the first bone portion relative to the second bone portion across a cut interface between the first bone portion and the second bone portion with a staple;
wherein fixating the first bone portion relative to the second bone portion with the staple comprises:
positioning a first leg of the staple connected to an inserter in a first implant hole of the first bone portion and positioning a second leg of the staple connected to the inserter in a second implant hole of a second bone portion, wherein the inserter is connected to the staple through a top surface of the staple;
positioning a convex bottom surface of a bridge connecting the first leg of the staple to the second leg of the staple against a concave surface of the proximal phalanx across the cut interface with a concave top surface of the bridge facing away from the proximal phalanx; and
detaching the inserter from the staple.

2. The method of claim 1, wherein positioning the convex bottom surface of the bridge against the concave surface of the proximal phalanx comprises positioning the convex bottom surface of the bridge against the concave surface that is a medial side of the proximal phalanx.

3. The method of claim 1, wherein positioning the convex bottom surface of the bridge against the concave surface of the proximal phalanx comprises positioning the convex bottom surface at each of a concave curvature at a surface of a metaphysis portion of the first bone portion and a concave curvature at a surface of a metaphysis portion of the second bone portion.

4. The method of claim 1, wherein the convex bottom surface of the bridge extends from the first bone portion to the second bone portion.

5. The method of claim 1, wherein:
the convex bottom surface of the bridge defines a local maximum point; and
positioning the convex bottom surface of the bridge against the concave surface of the proximal phalanx comprises positioning the local maximum point of the convex bottom surface at the cut interface.

6. The method of claim 1, further comprising applying a load force at the staple using the inserter, wherein, as the load force is applied at the staple using the inserter, the first leg and the second leg are caused to move apart from one another.

7. The method of claim 6, further comprising removing the load force from the staple (i) after positioning the first leg in the first implant hole and the second leg in the second implant hole, (ii) after positioning the convex bottom surface of the bridge against the concave surface of the proximal phalanx, and (iii) prior to detaching the inserter from the staple.

8. The method of claim 7, wherein removing the load force from the staple causes the first leg and the second leg to move toward one another to apply a compression force at the first bone portion and the second bone portion while maintaining the convex bottom surface and the concave top surface of the bridge.

9. The method of claim 6, wherein:
the inserter comprises a first coupling shaft connected through the top surface of the staple at the first leg and a second coupling shaft connected through the top surface of the staple at the second leg; and
applying the load force comprises biasing the first coupling shaft and the second coupling shaft toward each other.

10. The method of claim 9, wherein a connector joins the first coupling shaft and the second coupling shaft to bias the first coupling shaft and the second coupling shaft toward each other and maintain the load force at the staple while the connector joins the first coupling shaft and the second coupling shaft.

11. The method of claim 10, further comprising removing the connector from the first coupling shaft and the second coupling shaft to cause the first coupling shaft to move away from the second coupling shaft while maintaining the convex bottom surface and the concave top surface of the bridge.

12. The method of claim 1, wherein the convex bottom surface of the bridge defines an angle of curvature that is a same angle of curvature as the concave surface of the proximal phalanx across against which the bridge is positioned.

13. A system comprising:
a staple comprising a first leg on a first side of the staple, a second leg on a second side of the staple, a bridge connecting the first leg and the second leg, a first handling coupling extending through a top surface of the staple on the first side of the staple, and a second handling coupling extending through the top surface of the staple on the second side of the staple, the staple including a bottom surface configured to face one or more bones, the top surface of the staple being opposite the bottom surface of the staple, wherein the bridge includes a convex bottom surface that extends along at least a portion of the bottom surface of the staple at the bridge; and
an inserter configured to couple to the first handling coupling through the top surface of the staple and to the second handling coupling through the top surface of the staple,
wherein, when the inserter is connected to the first handling coupling and to the second handling coupling, the inserter is configured to apply a load force at the staple to cause the first leg and the second leg to move apart from one another.

14. The system of claim 13, wherein the convex bottom surface at the bridge corresponds to a curvature at a surface of a phalanx bone.

15. The system of claim 14, wherein the convex bottom surface at the bridge matches a concave curvature at a surface of a metaphysis portion of the phalanx bone.

16. The system of claim 15, wherein the convex bottom surface defines a local maximum point at a central portion along a length of the bridge.

17. The system of claim 16, wherein the local maximum point is along the length of the bridge so as to be spaced equally from each of the first handling coupling and the second handling.

18. The system of claim 15, wherein a length of the bridge between the first leg and the second leg is configured to span across a cut at the surface of the metaphysis portion of the phalanx bone.

19. The system of claim 15, wherein the inserter is configured such that when the inserter is removed from at least one of the first handling coupling and the second handling coupling the load force is removed from the staple to cause the first leg and the second leg to move toward one another to apply a compression force.

20. A system comprising:
a staple comprising a first leg on a first side of the staple, a second leg on a second side of the staple, a bridge connecting the first leg and the second leg, a first handling coupling extending through a top surface of the staple on the first side of the staple, and a second handling coupling extending through the top surface of the staple on the second side of the staple, the staple including a bottom surface configured to face one or more bones, the top surface of the staple being opposite the bottom surface of the staple, wherein the bridge includes a convex bottom surface that extends along at least a portion of the bottom surface of the staple at the bridge;
a first coupling shaft configured to couple to the first handling coupling, a second coupling shaft configured to couple to the second handling coupling, and a connector configured to join the first coupling shaft and the second coupling shaft,
wherein, when the connector is removed from at least one of the first coupling shaft and the second coupling shaft, the staple is configured such that the first leg and the second leg move toward one another while maintaining the convex bottom surface at the bridge.
